(19) United States
(12) Plant Patent Application Publication
(10) Pub. No.: US 2013/0283489 P1
(43) Pub. Date: Oct. 24, 2013

(54) Gentiana plant named 'KLEGH11002'

(50) ;

(71) Applicants: Nils KLEMM, Stuttgart (DE); Guido VON TUBEUF, Stuttgart (DE)

(72) Inventors: Nils KLEMM, Stuttgart (DE); Guido VON TUBEUF, Stuttgart (DE)

(73) Assignee: KLEMM+SOHN GMBH & CO. KG, Stuttgart (DE)

(21) Appl. No.: 13/506,450

(22) Filed: Apr. 19, 2012

(51) Int. Cl. A01H 5/00

(52) U.S. Cl. PLT/433

(57) ABSTRACT

A new variety of gentiana named ‘KLEGH11002’ particularly characterized by small leaves, very good branching, and medium early flowering time is disclosed.

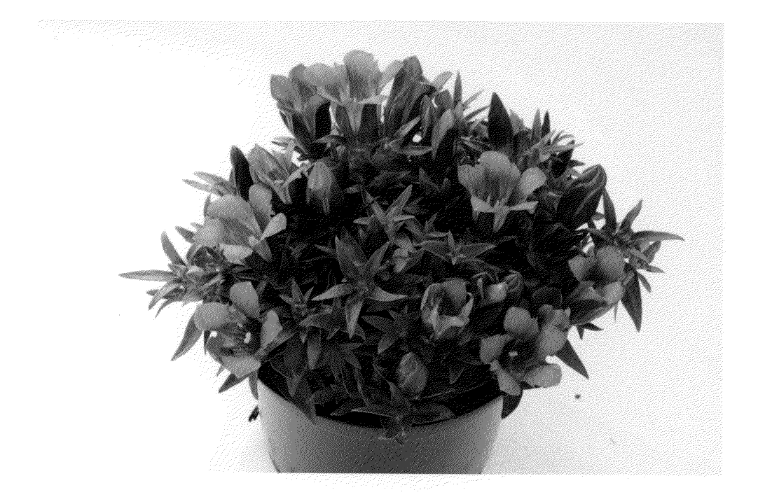

### GENUS AND SPECIES

Gentiana scabra

### VARIETY DENOMINATION

‘KLEGH11002’

### BACKGROUND OF THE NEW PLANT

The present invention comprises a new and distinct variety of gentiana, botanically known as Gentiana scabra, and hereinafter referred to by the variety name ‘KLEGH11002’. ‘KLEGH11002’ resulted from an open pollination in October 2003 in Stuttgart, Germany among a group of unnamed Gentiana scabra plants. A single plant selection was subsequently chosen for further evaluation and asexual propagation.

The new variety was first propagated via vegetative cuttings and in-vitro propagation by tissue culture in April 2009 in Stuttgart, Germany and has been asexually reproduced repeatedly by vegetative cuttings in Stuttgart, Germany for ten generations. ‘KLEGH11002’ has been found to retain its distinctive characteristics through successive asexual propagations via vegetative cuttings.

Plant Breeder's Rights for this variety have not been applied for. ‘KLEGH11002’ has not been made publicly available more than one year prior to filing of this application.

### SUMMARY OF THE INVENTION

The following are the most outstanding and distinguishing characteristics of this new variety when grown under normal horticultural practices in Stuttgart, Germany.

1. Medium early flowering time;

2. Very good branching; and

3. Small leaves.

### DESCRIPTION OF THE PHOTOGRAPH

This new gentiana plant is illustrated by the accompanying photograph which shows overall plant habit including flowers, flower buds, and foliage. The colors shown are as true as can be reasonably obtained by conventional photographic procedures. The photograph was taken in August 2010 on a 17-week old plant grown for 4 weeks from rooted cuttings in a 12 centimeter pot in a glass greenhouse in Stuttgart, Germany and subsequently grown outdoors for 13 weeks in Stuttgart, Germany under normal horticultural practices.

### DESCRIPTION OF THE NEW VARIETY

The following detailed descriptions set forth the distinctive characteristics of ‘KLEGH11002’. The data which define these characteristics were collected from asexual reproductions carried out in Stuttgart, Germany. The plant history was taken in the summer of 2011 on 17-week old plants grown in 12 centimeter pots in a glass greenhouse and outdoors with a pinch date at week 17. The color readings were determined under natural light. Color references are to the R.H.S. Colour Chart of The Royal Horticultural Society of London (R.H.S.), Fifth Edition (2007).

### DETAILED BOTANICAL DESCRIPTION OF THE NEW PLANT

Classification:
   Family.—Gentianaceae.
   Genus and species.—Gentiana scabra.
   Common name.—Gentiana, Japanese gentiana.
   Denomination.—‘KLEGH11002’.
Plant:
   Form.—Rounded.
   Habit.—Upright to mounding and arching.
   Height (from top of soil).—6.0 cm to 8.0 cm.
   Width (horizontal plant diameter).—8.0 to 10.0 cm.
   Propagation.—Terminal vegetative cuttings and tissue culture.
   Time to produce a finished flowering plant.—16 to 18 weeks.
   Time to initiate and develop roots.—4 to 5 weeks.
   Root description.—Fine.
   Root color.—RHS N155C.
Stems:
   Average quantity of basal branches.—3 to 4.
   Length of basal branches (from the base of the stem to the tip).—Approximately 12.0 cm.
   Internode length.—1.0 cm to 2.0 cm.
   Diameter of branches (from midpoint).—Approximately 0.3 cm.
   Stem color.—RHS 145A and becoming reddish (RHS N34C) above the leaf pairs.
   Texture.—Smooth.
Leaves:
   Arrangement.—Opposite.
   Length.—2.0 cm to 3.0 cm.
   Width.—Approximately 1.0 cm.
   Shape.—Lanceolate.
   Margin.—Entire.
   Apex.—Acute.
   Base.—Rotundate.
   Color.—Immature: Upper surface: RHS 135C Lower surface: RHS 136C Mature leaf: Upper surface: Between RHS 135A and RHS 137A Lower surface: RHS 136C.
   Texture (both surfaces).—Smooth.
   Venation pattern.—Pinnate.
   Venation color (both surfaces).—RHS 136C.
   Petioles.—Absent, leaves fused at base.
Inflorescence:
   Blooming habit (flowering season).—Late summer.
   Inflorescence type.—Single terminal flowers.
   Lastingness of individual inflorescence on the plant.—Approximately 1 to 2 weeks.
   Fragrance.—Absent.
   Pedicels.—Absent.
   Peduncles.—Absent.
Flower bud:
   Shape.—Oblong.
   Length.—3.5 cm to 4.0 cm.
   Diameter.—0.8 cm to 1.0 cm.
Flowers:
   Quantity per plant.—5 to 10.
   Arrangement.—Single, terminal.
   Shape.—Campanulate.
   Petals.—Quantity per flower: 5 Arrangement: Fused.
   Sepals.—Quantity per flower: 5 Shape: Lanceolate Apex: Acute Base: Fused Margin: Entire.
Reproductive organs:
   Stamens.—Quantity: 5.
   Pistils.—Quantity: 1.
Fruit and seed set: None observed.
Disease, pest, and stress resistance: No particular resistance observed.
Comparison with commercial variety: ‘KLEGH11002’ differs from the commercial variety ‘Alpine Success Blue 05’ (U.S. Plant Pat. No. 18,767) in that ‘KLEGH11002’ has a medium early flowering time, whereas ‘Alpine Success Blue 05’ flowers one to two weeks later than ‘KLEGH11002’. Additionally, ‘KLEGH11002’ has smaller leaves than ‘Alpine Success’.

We claim:

1. A new and distinct variety of gentiana plant named ‘KLEGH11002’ as shown and described herein.
